(19) United States
(12) Patent Application Publication
(10) Pub. No.: US 2007/0065826 A1
(43) Pub. Date: Mar. 22, 2007

(54) Cationic dye compound for detecting nucleic acid double strand, and detection method and apparatus using same

(75) Inventors: Chikako Takatoh, Kanagawa (JP); Kazuyoshi Takeda, Kanagawa (JP); Hirokazu Sato, Kanagawa (JP); Takayuki Saito, Kanagawa (JP); Takuya Matsumoto, Kyoto (JP); Tomoji Kawai, Osaka (JP)

Correspondence Address: BROWDY AND NEIMARK, P.L.L.C.;624 NINTH STREET, NW, SUITE 300, WASHINGTON, DC, 20001-5303, US

(73) Assignee: EBARA CORPORATION, TOKYI (JP)

(21) Appl. No.: 10/570,594

(22) PCT Filed: Sep. 6, 2004
(86) PCT No.: PCT/JP04/13264

§ 371 Date: Oct. 31, 2006

(30) Foreign Application Priority Data
Sep. 5, 2003 (JP) 2003-314662

(51) Int. Cl. C12Q 1/68; C09B 47/04; C07D 487/22; C07H 21/04

(52) U.S. Cl. 435/6; 536/25.32; 540/125; 540/145

(57) ABSTRACT

A cationic dye compound having novel uses, the cationic dye compound being capable of conveniently and rapidly detecting a nucleic acid double strand, and a detection method using the cationic dye compound are provided. This invention relates to a cationic dye compound for detecting a nucleic acid double strand, comprising: a cation group and a chromophore coupled to said cation group, wherein said chromophore has a heteropolycyclic structure containing a nitrogen atom and said cationic dye compound is capable of helically binding onto said nucleic acid double strand.

### TECHNICAL FIELD

This invention relates to a cationic dye compound for detecting a nucleic acid double strand, such as a hybridization product of an oligonucleotide or a polynucleotide, and also relates to a method and an apparatus for spectroscopically detecting a nucleic acid double strand in a sample by use of the cationic dye compound.

### BACKGROUND ART

In gene analysis based on the hybridization method, it has been common practice to cause a target nucleic acid to interact with a probe nucleic acid, extract the target nucleic acid complementary in sequence to the probe nucleic acid, and detect the target nucleic acid. Normally, the target nucleic acid is labeled with a detectable group, and the existence of a hybrid is detected, whereby the presence or absence of the target nucleic acid, or the amount of the target nucleic acid present is analyzed. With the hybridization method, a labeling method using a radioisotope, or a labeling method using a fluorescent substance, as the detectable group, is known. However, preparation for labeling with the detectable group beforehand results in a complicated procedure. Furthermore, detection with the radioisotope takes time, involves difficult analysis, and presents problems about the safety and cost of handling. Detection with the fluorescent substance, on the other hand, is excellent in safety and the rapidity of detection. However, this method poses the problems that the fluorescent substance is expensive, and that the uptake of the fluorescent substance into the target nucleic acid is low.

Besides, a detection method using an intercalator is available as a convenient method which does not need the previous labeling with the detectable group. Known intercalators are those which exhibit electrochemical activity, or emit fluorescence, upon insertion into the complementary nucleic acid. Japanese Patent Application Laid-Open No. 1997-288082 discloses a method for detecting a target nucleic acid by measuring the amount of an electric current flowing between an electrochemical activity threading intercalator and an electrode provided with an output terminal, the electrochemical activity threading intercalator being bound to a hybrid nucleic acid formed from a nucleic acid fragment immobilized onto the electrode and the target nucleic acid. However, this electrochemical detection method requires the surface treatment of the electrode for improving sensitivity. Japanese Patent Application Laid-Open No. 2001-289848 shows a method which detects fluorescence given off when a fluorescent intercalator is inserted and bound between base pairs of a hybrid nucleic acid. Generally, the insertion and elimination of an intercalator into and from a hybrid nucleic acid in an aqueous solution are in an equilibrium relationship. Thus, fluorescence from the intercalator in the eliminated state serves as a background for detection. Its inventor utilizes the enhancement effect of a fluorescent substance whose fluorescence is increased only when bound to a nucleic acid. However, the enhancement effect due to binding is not limited to a double-stranded nucleic acid fragment, but occurs for a single-stranded nucleic acid fragment as well. The rate of dissociation between the single-stranded nucleic acid fragment and the fluorescent intercalator is high compared with that for the double strand. However, fluorescence from the intercalator bound to the single-stranded nucleic acid provides a background for fluorescence detection, lowering the S/N ratio.

With the conventional Southern hybridization technique, improvements in sensitivity have been achieved by using an electrochemical activity threading intercalator or a fluorescent intercalator from the viewpoint of convenience and rapidity. However, there are limits on the proportion of insertion of the intercalator into base pairs, making it difficult to increase sensitivity. Moreover, the proportion of intercalator insertion is affected by the sequence of base pairs, thus leading to poor determinability. Furthermore, the insertion and elimination of the intercalator into and from the hybrid complex in an aqueous solution are in the equilibrium relationship. Thus, fluorescence from the fluorescent intercalator in the eliminated state, or fluorescence from the intercalator bound to a single-stranded nucleic acid, according to the fluorescence method, brings about the drawback that a sufficient S/N ratio cannot be achieved.

As described above, various methods of detection have been studied. However, all of these methods pose the following problem of complicacy in addition to the aforementioned defects: To detect the presence or absence of a particular gene sequence, DNA taken out of a sample has to be fragmented with a restriction enzyme, and then fractionated according to size by a method such as electrophoresis, where after the test DNA must be immobilized onto nitrocellulose paper or the like.

The present invention aims at solving the above-described problems. The object of the present invention is to provide a cationic dye compound having novel uses, the cationic dye compound being capable of conveniently and rapidly detecting a nucleic acid double strand, such as a hybrid of a nucleic acid probe and a target nucleic acid complementary to the nucleic acid probe, and to provide a detection method using the cationic dye compound, and so on.

### BRIEF DESCRIPTION OF THE DRAWINGS

FIG. 1a is a view illustrating the molecular structures of cationic dye compounds according to the present invention.

FIG. 1b is a view illustrating the methods of synthesizing the cationic dye compounds of the present invention.

FIG. 2 is a schematic drawing showing a double-stranded DNA to which the cationic dye compound of the present invention has been bound.

FIG. 3 is a graph showing the CD spectrum of a sample containing only the double-stranded DNA.

FIG. 4 is a graph showing the CD spectrum of a sample containing only a porphyrin derivative cationic dye compound.

FIG. 5 is a graph showing the CD spectrum of a sample containing both of the double-stranded DNA and the porphyrin derivative cationic dye compound.

FIG. 6 is a graph showing the CD spectrum of a sample containing only a phthalocyanine derivative cationic dye compound.

FIG. 7 is a graph showing the CD spectrum of a sample containing both of the double-stranded DNA and the phthalocyanine derivative cationic dye compound.

### SUMMARY OF THE INVENTION

The inventor diligently conducted studies in an attempt to solve the aforementioned problems, and realized that when a cationic dye compound is bound onto a nucleic acid double-stranded structure, its spectroscopic properties (including identification chirality (optical activity) and wavelength shift) are exhibited or changed. The inventor found that the specific detection of a nucleic acid double strand can be attained by measuring, for example, circular dichroism.

That is, the present invention relates to a cationic dye compound for detecting a nucleic acid double strand, comprising: a cation group and a chromophore coupled to said cation group, wherein said chromophore has a heteropolycyclic structure containing a nitrogen atom and said cation group is capable of binding to a double stranded nucleic acid.

The cationic dye compound in a preferred embodiment is represented by the following general formula (I):
X—(Y-Z)ₙ (I)
where n denotes 1 to 12, X represents a choromophore having at least four pyrrole rings, Y represents a connecting group or a direct bond between X and Z, and Z represents a cationic functional group, or a functional group whose property is convertible to a cationic property.

The preferred cationic dye compound has the choromophore which is selected from the group consisting of porphyrin, porphyrin derivatives, phthalocyanine, and phthalocyanine derivatives.

The method of the present invention, which detects a hybrid nucleic acid by use of a cationic dye compound, is a method comprising:

providing a cationic dye compound comprising a cation group and a chromophore coupled to said cation group, said chromophore having a heteropolycyclic structure containing a nitrogen atom;

bringing a nucleic acid probe and a sample containing a target nucleic acid into contact with each other under hybridization conditions to form a hybrid nucleic acid composed of said nucleic acid probe and said target nucleic acid;

binding said cationic dye compound onto said hybrid nucleic acid by adding the cationic dye compound before, during or after said hybridization; and

measuring spectroscopic properties of said cationic dye compound bound onto said hybrid nucleic acid.

In the method of the present invention, the cationic dye compound can be represented by the following general formula (I):
X—(Y-Z)ₙ (I)
where n denotes 1 to 12, X represents a choromophore having at least four pyrrole rings, Y represents a connecting group or a direct bond between X and Z, and Z represents a cationic functional group, or a functional group whose property is convertible to a cationic property.

Further, the method of the present invention can also comprise immobilizing an analyte having said target nucleic acid or said nucleic acid probe onto a solid phase carrier to bring said analyte and said nucleic acid probe into contact with each other under hybridization conditions.

The present invention also provides an apparatus for detecting a hybrid nucleic acid by use of a cationic dye compound, comprising:

means for bringing a nucleic acid probe and a sample containing a target nucleic acid into contact with each other under hybridization conditions to form a hybrid nucleic acid composed of said nucleic acid probe and said target nucleic acid;

means for binding said cationic dye compound onto said hybrid nucleic acid by adding the cationic dye compound before, during or after said hybridization, wherein said cationic dye compound comprises a cation group and a chromophore coupled to said cation group, said chromophore having a heteropolycyclic structure containing a nitrogen atom; and

means for measuring spectroscopic properties of said cationic dye compound bound onto said hybrid nucleic acid.

Definition of Terminology

The term “nucleic acid” used herein may be DNA, RNA, or a natural or artificial polynucleotide which is an analogue thereto. More particularly, the nucleic acid includes all oligonucleotides or polynucleotide derivatives or their analogues as long as they can form a double strand based on complementarity between base sequences.

The term “nucleic acid double strand”, as used herein, refers to a duplex nucleic acid structures formed from analogous or heterogeneous nucleic acids as stated earlier. More particularly, the nucleic acid double strand includes any nucleic acid structures involving other higher-order structures, as long as they allow the cationic dye compound of the present invention to be bound to at least some phosphate groups of the higher-order structure, and permit the binding products to be regularly juxtaposed on the helical structure.

The above-mentioned nucleic acid double strand may itself be an analyte, or may be part of a detection object or sample, such as a biological component, a biological substance, or other low molecular weight substance or polymeric substance. For example, the nucleic acid double strand may be, although not limited to, any products coupled with other nucleic acids, nucleotides, and oligonucleotides, or maybe complexes with high molecular weight substances, such as hormones, enzymes, proteins, oligopeptides, and polysaccharides, or complexes with low molecular weight substances, such as medications, steroids, and sugars. The types of samples containing such analytes are not limited. Their examples include biological samples separated and collected from mammals including humans, such as blood, urine, sweat, tissue pieces, organ pieces, and hair. These analytes may be detected in vivo, in vitro, or ex vivo.

The term “circular dichroism” or “CD”, as used herein, refers to the property that incident plane-polarized light changes into elliptically polarized light by passing through a analyte having chirality. This property can be expressed as the angle of optical rotation which varies with a wavelength for measurement. Generally, circular dichroism is obtained as molar ellipticity (=Mθ/(lC) where θ is the elliptic angle, l is the cell length, C is the concentration, and M is the molecular weight).

### DETAILED DESCRIPTION OF THE INVENTION

The cationic dye compound in the preferred embodiment of the present invention is a compound represented by the general formula (I) shown below. That is, the cationic dye compound has the choromophore X, and at least one cationic functional group Z coupled thereto.
X—(Y-Z)ₙ (I)
where n denotes 1 to 12, X represents a choromophore, Y represents a connecting group or a direct bond between X and Z (i.e., which can be optionally used), and Z represents a cationic functional group, or a functional group whose property is convertible to a cationic property.

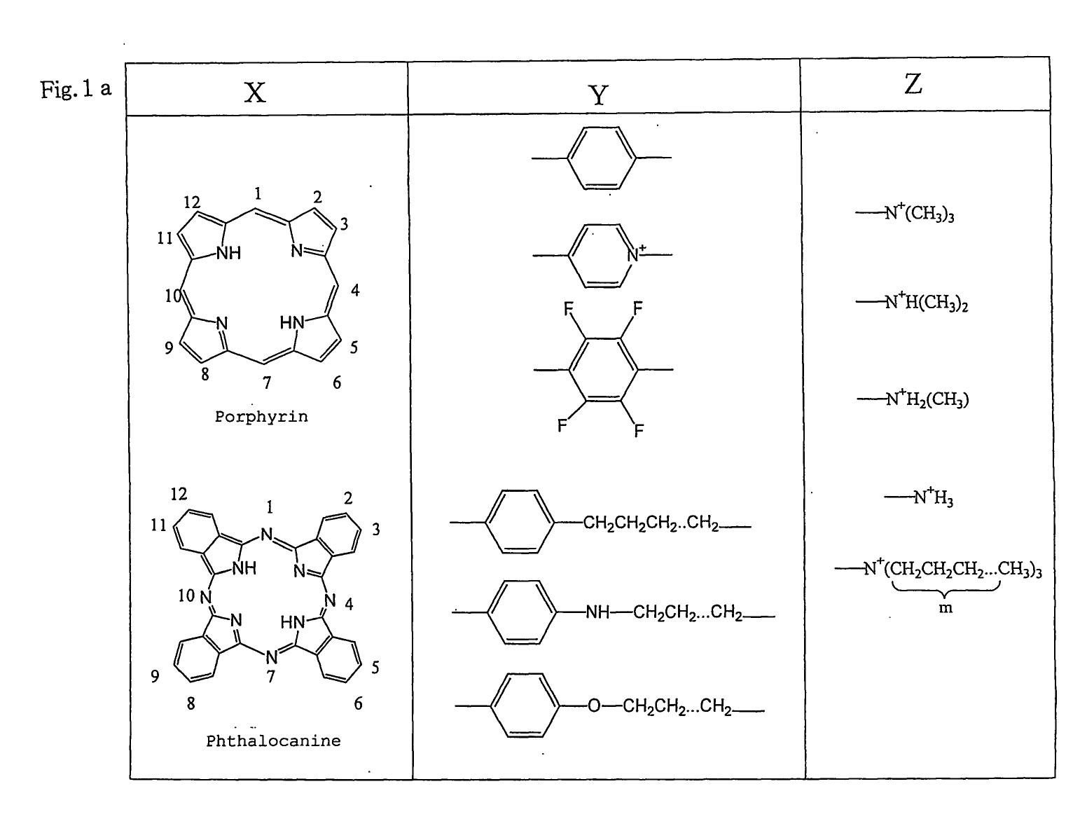

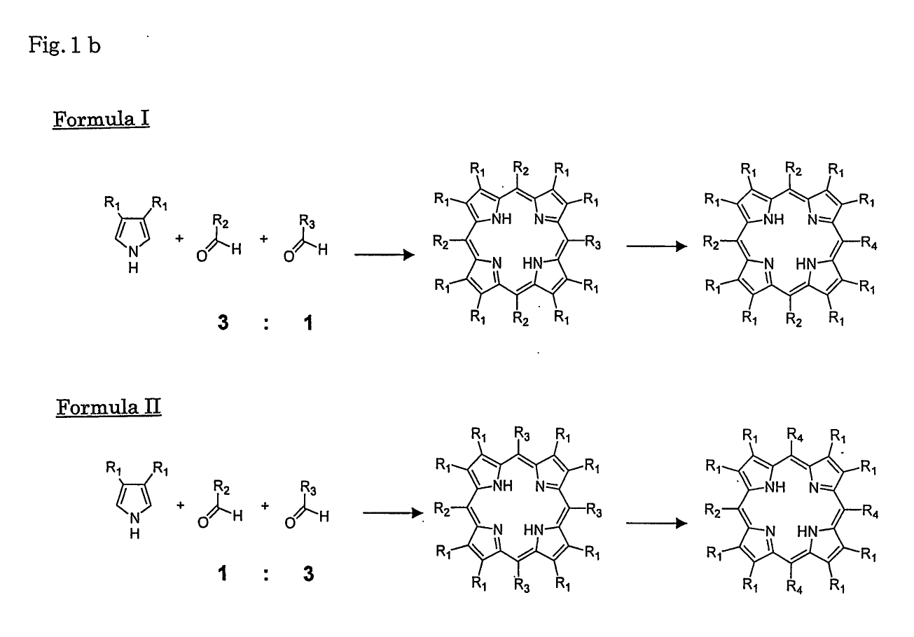

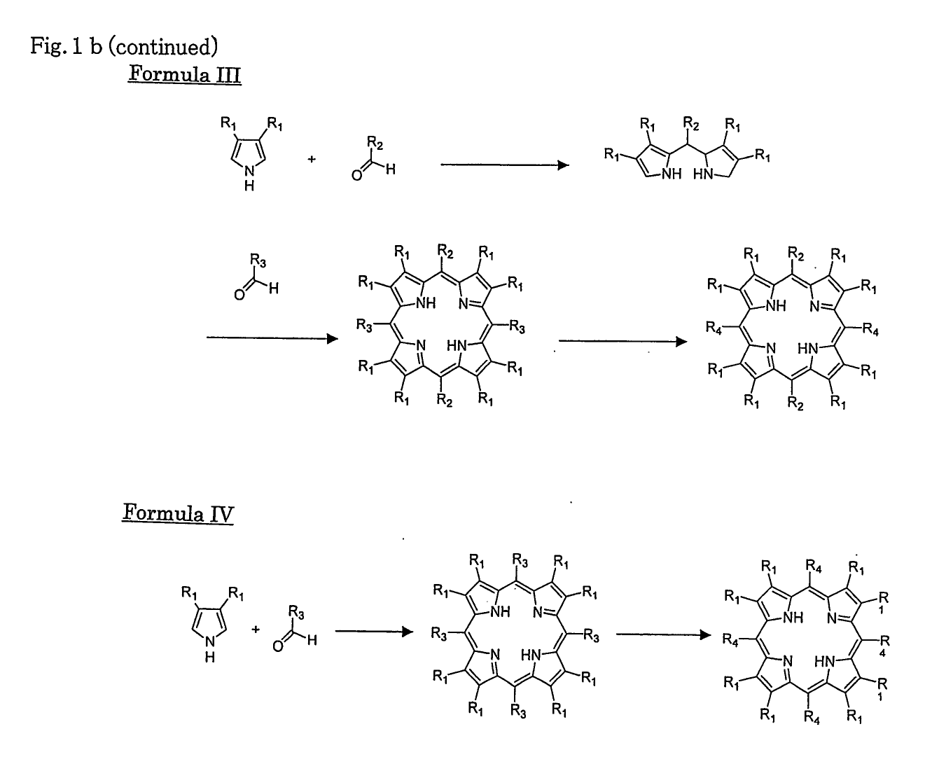

Each of the structural portions in the above formula will be concretely described, but the cationic dye compound of the present invention is not limited to those shown in FIG. 1.

The choromophore X is a core portion of the cationic dye compound of the present invention, and may be a cyclic group having planarity, or an aromatic group having a condensed ring. Particularly preferably, the choromophore X is an aromatic group of a heteropolycyclic structure containing nitrogen atoms, as shown in FIG. 1a. Provided that water solubility can be ensured, the planarity of the choromophore preferably spreads greatly two-dimensionally. The choromophore X may also have various substituents on such a heteropolycyclic skeletal structure. Particularly preferred as the choromophore X of the heteropolycyclic structure is a choromophore having at least four pyrrole rings, for example, porphyrin, a porphyrin derivative, phthalocyanine, or a phthalocyanine derivative, and a central metal may be present or absent. As regards the characteristics of such a choromophore X, the preferred choromophore is a choromophore having absorption in the ultraviolet (UV) and visible light (VIS) regions, for example, like porphyrin. Further, the choromophore preferably has absorption in a region which does not overlap a region in the vicinity of 260 nm, the absorption zone for nucleic acids. Particularly, porphyrin, phthalocyanine, a porphyrin derivative, or a phthalocyanine derivative is preferred, because its absorption region contains absorption of the Soret band at a wavelength in the vicinity of 400 nm, and absorption of the Q-band at a wavelength in the vicinity of 600 nm to 700 nm. Moreover, the choromophore with the Soret band and the Q-band is preferred in that its absorption wavelength can be adjusted to a long wavelength side and a short wavelength side by modification.

The cationic functional group Z represents a cationic functional group, or a functional group whose property is convertible to cationic property, as shown in FIG. 1a. Such a cationic functional group is typified by an onium of each of —N, —C, —S, —P, —I and —O. The onium refers to a substance represented by a chemical formula, such as a phosphonium [R₃PR′]⁺, an oxonium [R₂OR′]⁺, an iodonium [RIR′]⁺, or a sulfonium [R₂SR′]⁺. As the cationic functional group, a quaternary (tetraalkyl) ammonium group, or a functional group convertible to a quaternary ammonium group is particularly preferred. Examples of a substituted quaternary ammonium group are those of the general formula —N⁺R1R2R3 (where R1, R2 and R3 each represent —(CH₂)ₘCH₃, with m denoting 0 to 20, preferably 0 to 10 (H in the case of 0), more preferably 0 to 4). Other substituted quaternary ammonium groups include, for example, those of the general formula —N⁺R1R2(C₂H₄O)_{q}R3 (where q denotes 1 to 4), such as polyethylene glycol. The number of the functional groups Z is 1 to 12, preferably 1 to 8, more preferably 1 to 4, even more preferably 2 or more in each range, per dye. Assume that the choromophore is porphyrin, for example. If, in the porphyrin of FIG. 1a, the cationic functional groups Z are bound to all of the four meso-locations at the 1-, 4-, 7- and 10-positions, the number of the functional groups Z is 4 per choromophore. If the functional groups Z are bound to two meso-positions in the symmetric relationship or in the mirror-image relationship, the number of the functional groups Z is 2 per choromophore. If, in the porphyrin of FIG. 1a, the cationic functional groups Z are bound to all of the eight β-locations at the 2-, 3-, 5-, 6-, 8-, 9-, 11- and 12-positions, the number of the functional groups Z is 8 per choromophore. If, in the porphyrin of FIG. 1a, the cationic functional groups are bound to all of the meso-positions and the β-positions, the number of the functional groups Z is 12 per choromophore.

The connecting group Y is to connect the X and Z described above, and has a skeletal backbone chain comprising a carbon, oxygen and/or nitrogen atom optionally having substitutions, or a cyclic structure, as shown in FIG. 1a. However, the connecting group Y is an arbitrary factor for the present invention, and the choromophore X may be directly bonded to Z.

The connecting group Y plays the role of optimizing the physical distance between the choromophore X and the functional group Z. Optimization of the distance between X and Z refers to adjustment of the distance from the choromophore X to X (normally, it is considered preferred that this distance corresponds to the pitch between the phosphate groups of the nucleic acid). The distance depends on the size of the choromophore X as well. With a relatively large choromophore as shown in FIG. 1a, for example, the plural binding sites of the functional group X may, advantageously, have a distance similar to the pitch between the phosphate groups of the nucleic acid. In such a case, the connecting group Y is considered to be unnecessary, or maybe of a relatively short length. A typical connecting group includes a phenylene group, an alkylene group, or a combination of them.

In consideration of the above-mentioned aspects and the ease of synthesis, in an example of porphyrin in which the choromophore X is bound to four functional groups Z at the meso-positions, the connecting group Y is preferably a single phenylene group. In the case of porphyrin, the aforementioned —Y-Z group may be further bound to the eight beta-positions. An alkylene group has the effect of stabilizing the stack structure of the choromophore X, and affording an improvement in solubility in a solvent. The number of the carbon atoms in its backbone chain is 0 to 100, preferably 0 to 20. Any person skilled in the art would be able to design and synthesize, in the usual manner, all types of the connecting group Y and the cationic group suitable for the size of the choromophore X, the locations of the cationic functional groups Z, and so on, and achieve the binding positions and the number of these groups. All of these embodiments are within the scope of the present invention.

If the connecting group Y has an extensive structure, such as an aromatic ring, a condensed ring, or a heterocyclic ring, there may be a case where any of them has a solid angle with respect to the planes of the conjugated system of the choromophore. This is useful as a steric hindrance for preventing the intercalation of the choromophore X between base pairs having phosphate groups. Such an intercalation between base pairs will be a factor impeding the detection of the circular dichroism of the choromophore X, so that it may be preferred for the connecting group Y to have such a structure. As such a connecting group Y, a phenylene group, an alkylene group, a pyridylene group, or any combination of them is named. According to the inventor's study, the cationic dye compound in one embodiment, which is considered to be particularly preferred, includes a structure in which one phenylene group, as the connecting group Y, is present at each of the four meso-positions of porphyrin, and the cation group Z is bound to two of these adjacent phenylene groups.

As shown in FIG. 1a, preferred examples of the cationic dye compound are those which have phenylene groups at the meso-positions of porphyrin, and have quaternary ammonium groups, as the functional groups Z, at the phenylene groups. Preferred as the connecting group Y extending from porphyrin is a phenylene group incorporating the functional group Z, or a single phenylene group, or that having a further alkylene group (optionally having an alkoxy residue or an amide residue partly) between a phenylene group and the functional group Z. The cationic dye compound may be a commercially available one, or can be obtained by synthesis. The cationic dye compound having a general porphyrin skeleton can be synthesized using a general method of synthesis described in the literature (literature: The Porphyrin Handbook; Kadish, K. M., Smith, K. M., Guilard, R., Eds.; Academic Press: San Diego, 2000; Vol. 1). An outline of synthesis is shown in Formulas I to IV of FIG. 1b. Formula I shows the method of synthesizing the cationic dye compound having only one cationic functional group R4, Formula II shows the method of synthesizing the cationic dye compound having three cationic functional groups R4, Formula III shows the method of synthesizing the cationic dye compound having two cationic functional groups R4, and Formula IV shows the method of synthesizing the cationic dye compound having four cationic functional groups R4. In Formulas I, II and IV, a pyrrole derivative and an aldehyde compound, which are starting materials, are reacted in the presence of an acid (trifluoroacetic acid, boron trifluoride etherate or the like is used preferably). Then, the resulting product is oxidized with an oxidizing agent (chloranil or 2,3-dichloro-5,6-dicyano-1,4-benzoquinone is used preferably) to obtain porphyrin as a cyclic compound. Then, R3, which is a cationic functional group precursor, is converted into a cationic functional group R4 by use of a publicly known method. In Formula III, a pyrrole derivative and an aldehyde compound, which are starting materials, are reacted in the presence of an acid (trifluoroacetic acid, boron trifluoride etherate or the like is used preferably) to synthesize dipyrromethane. Then, this product is reacted with an aldehyde compound having a cationic functional group precursor R3 in the presence of an acid, where after the resulting product is oxidized to obtain porphyrin as a cyclic compound. Then, R3, which is the cationic functional group precursor, is converted into a cationic functional group R4 by use of a publicly known method.

The cationic dye compound of the present invention can be used in the following manner:

The above-described cationic dye compound is ion-bonded and/or electrostatically bonded to a negatively charged portion on the surface of a nucleic acid duplex structure, at least, at the cation group, which is a part of the cationic choromophore, in an aqueous solution of a reaction system. The interaction between the thus formed at least two molecules of the dye compound adjacent to each other along the double helix is presumed to affect spectroscopic properties.

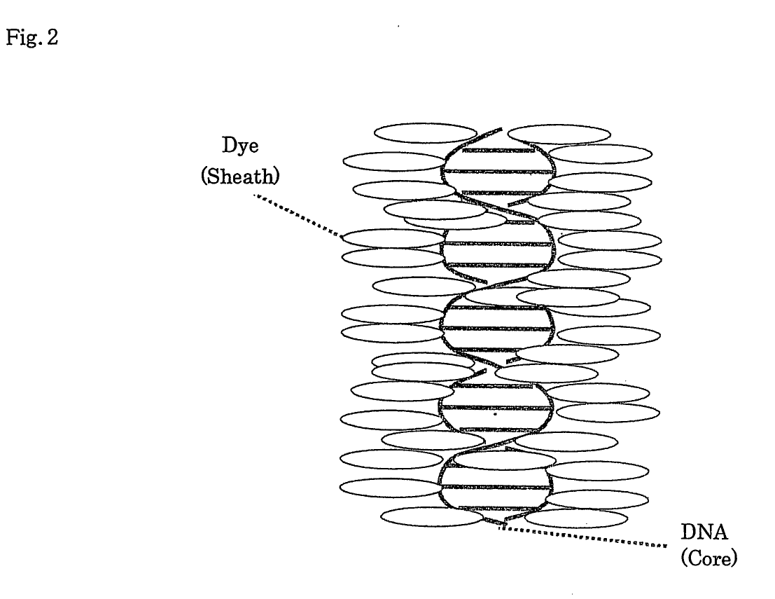

FIG. 2 shows the cationic dye compound bound to the nucleic acid double strand. In this drawing, the cationic dye compound can be ion-bonded to a negatively charged site, such as anion groups (typically, each phosphate group), of the nucleic acid double strand via each cation group. Thus, the cationic dye compound can form a core-sheath structure in which the cationic dye compound helically covers the periphery of the nucleic acid (for example, DNA) as a core. When the cationic dye constitutes the sheath portion of such a core-sheath structure, it generates characteristic chirality and/or a wavelength shift. As the characteristic chirality, circular dichroism and/or fluorescence polarization characteristics can be named. These spectroscopic characteristics rely on a physicochemical interaction between the dyes regularly adjacent to each other in the helical core-sheath structure.

Generally, a choromophore has no chirality or the like when it is in a monomolecular state. However, it is known that under the interaction between two or more molecules of the choromophore, an absorption wavelength region changes. A greater change in wavelength is more suitable for detection. According to the present invention, however, even the cationic choromophore having chirality in the monomolecular state forms the above-described helical configuration by binding to the nucleic acid double strand. Thus, a wavelength shift or characteristic chirality attributed thereto can be exhibited, thereby making it possible to determine whether the choromophore is one binding to the nucleic acid double strand.

As the method of detection to which the present invention is applicable, there can be named the hybridization technique for detecting a hybrid of a nucleic acid probe and a target nucleic acid, as typified by Southern hybridization. The cationic dye compound rendered coexistent under the hybridization conditions forms a helical heath structure covering the resulting hybrid nucleic acid, and a CD peak is observed in the absorption wavelength by a CD spectroscope. If no hybrid is formed, namely, if DNA remains to be a single strand, no helical core-sheath structure is formed. In this case, a circular dichroism peak or a wavelength shift does not appear in the absorption wavelength of the cationic choromophore.

The method or apparatus of the present invention, which involves a hybridization reaction, preferably has a step or means for heating a reaction solution and, if desired, can have a cooling step or means. That is, the reaction solution is heated to dissociate double-stranded DNA into single strands, which can then be hybridized with a complementary DNA probe. In such a hybridization reaction, the cationic dye compound may coexist, or may be added after the hybridization. The concentration of the cationic dye compound in the hybridization reaction solution is several nanomols/L to several micromols/L, preferably 2 μmols/L.

The method or apparatus of the present invention can be performed or used, with a reactant, such as the DNA probe or an analyte having the target DNA, being rendered free in the solution. Alternatively, there may be provided means for immobilizing the DNA probe or an analyte having the target DNA onto a solid carrier and, if desired, means for washing the solid carrier after the reaction, thereby removing a sample which has not been hybridized. As described earlier, a CD spectroscope can be used for detection of the cationic dye compound bound to the hybrid. At this time, single-stranded DNA and the unbound cationic dye compound can be detected as such, because they do not have a CD peak as observed with the cationic dye compound during sheath-structure formation. Hence, the present invention, whether it uses a solid carrier or not, has the advantage that a washing step prior to detection is unnecessary. In hybridizing the DNA or the analyte having thereof on the solid carrier in the reaction solution, it is advisable to provide a step of immobilizing DNA or the like onto the solid carrier before the hybridization reaction, as described above.

As the method of immobilizing DNA or the like onto the solid carrier, any method publicly known in the field of DNA chip production can be used. Any solid carrier, which can immobilize a nucleic acid or the like, can be used as the above solid carrier usable in the present method. The solid carrier is in the form of a substrate, beads, balls, or a sandwich. The method of detecting the sample on the solid carrier by CD includes a method in which irradiated light to induce CD is reflected on the surface of the solid carrier and detected, and a method in which irradiated light to induce CD is passed through the solid carrier and detected. When passed through by the light, the solid carrier is preferably transparent. Preferably, the solid carrier is glass or ITO. As the substrate when detection is performed by reflection, graphite, mica, or silicon wafer is named. Other examples of the solid carrier are porous materials, such as ceramics, polymer, cloth or paper, and beads.

As the nucleic acid probe, there can be employed DNA fragments formed by cleaving DNA extracted from a biological sample with the use of a restriction enzyme, and purifying the cleaved pieces, for example, by electrophoretic or other separation, or chemically synthesized DNA. Preferably, the nucleic acid probe is sequenced beforehand according to a well known sequencing method.

### EXAMPLE

(1) Detection of Binding of a Porphyrin Derivative Dye Compound to Double-Stranded DNA

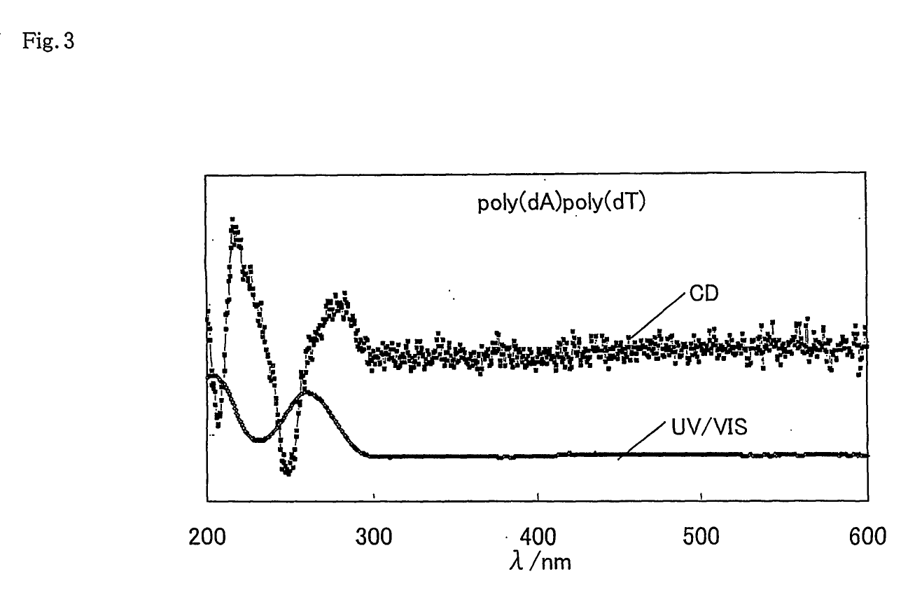

Synthetic DNA (Poly(dA)/Poly(dT), 50 mers.) used was that of Amersham Pharmacia Biotech with a concentration of 25 A₂₆₀ units and counter ions being those of a sodium salt. This DNA was adjusted to 0.125 unit with Milli-Q (registered trademark) water sterilized for 20 minutes at 120° C., was allowed to stand for 1 day, and was then measured for CD. A CD spectrum obtained showed a CD peak ascribed to the helical shape of the DNA at a wavelength in the vicinity of 260 nm (see FIG. 3).

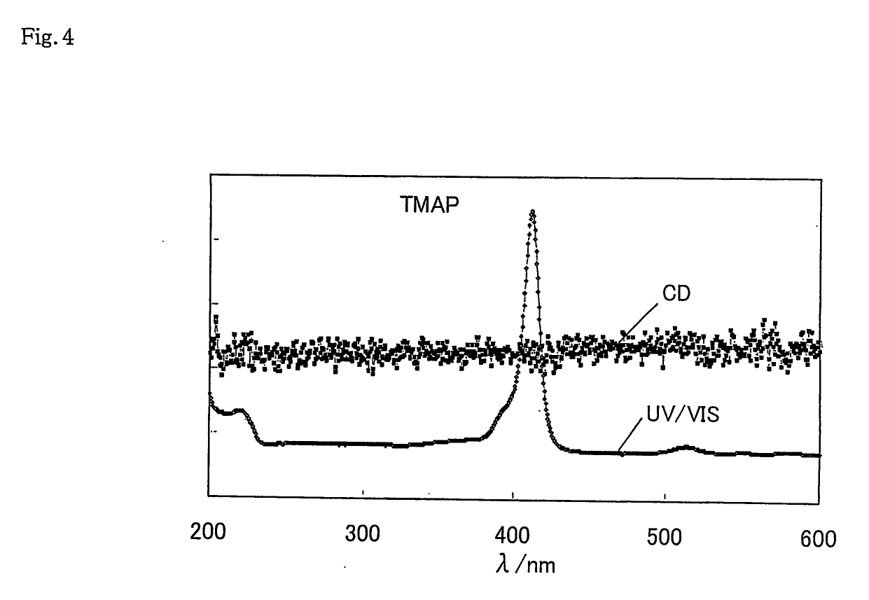

Next, a powder of tetrakis(4-N-trimethylamino-phenyl)porphine tetra(p-toluenesulfonate) (hereinafter referred to as TMAP), as a cationic choromophore, produced by FULKA was adjusted to an aqueous solution of 2×10⁻⁶ mol/L, and measured for CD. In a CD spectrum obtained, a CD peak showing chirality did not appear (see FIG. 4).

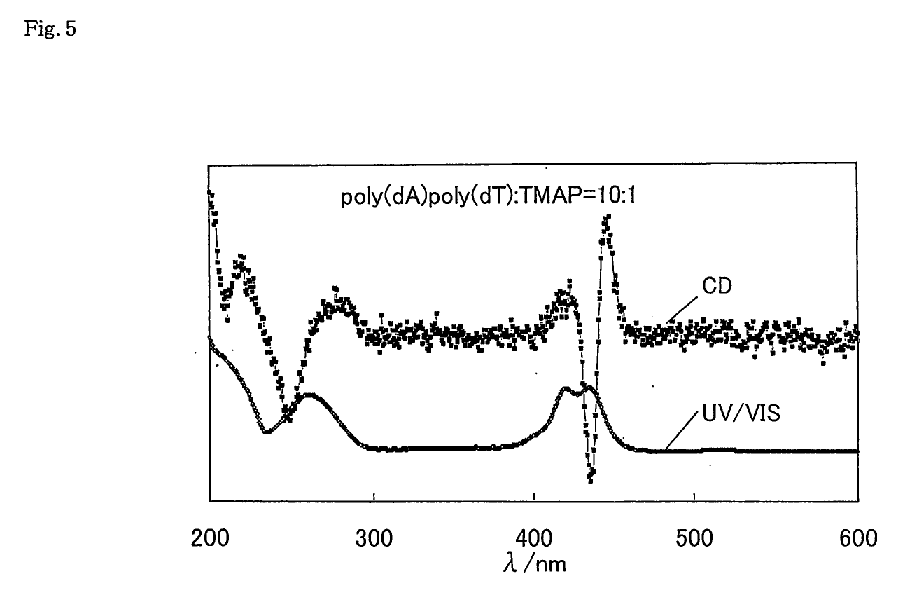

Next, 0.25 unit of Poly(dA)/Poly(dT) and 4×10⁻⁶ mol/L of TMAP were mixed in equal amounts, allowed to stand at room temperature, and measured for CD at a wavelength in the range of 200 to 600 nm (see FIG. 5). A sharp CD peak, which was not observed in the measurement of only DNA (FIG. 3) or only TMAP (FIG. 4), was confirmed at a wavelength in the vicinity of 410nm in the Soret band for TMAP. In such a region, the CD spectrum showed Cotton effect involving dextrorotation, and twisted alternately (vertically in the drawing), showing a TMAP-TMAP correlation reflecting a helical structure. The absorption peak of UV/VIS, measured at the same time, was shifted from the absorption wavelength of the Soret band, which was observed with only TMAP, to a long wavelength side because of mixing with DNA. Thus, according to “Kasha's theory”, the porphyrin skeleton of TMAP was confirmed to have a head-to-tail orientation, thus confirming that the CD peak was a CD peak attributed to the helical structure of TMAP.

(2) Detection of Binding of a Phthalocyanine Derivative Dye Compound to Double-Stranded DNA

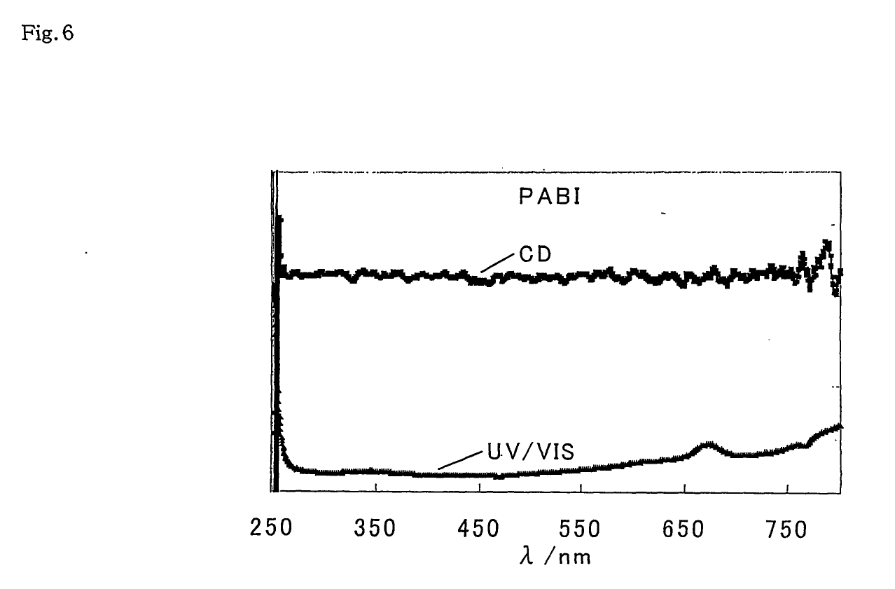

A powder of Propyl Astra Blue Iodide (hereinafter referred to as PABI), as a cationic choromophore, produced by SIGMA ALDRICH was adjusted to a solution of 2×10⁻⁶ mol/L with the use of a solvent (a product of SIGMA-ALDRICH), and measured for CD. In a CD spectrum obtained, a CD peak showing chirality did not appear (see FIG. 6).

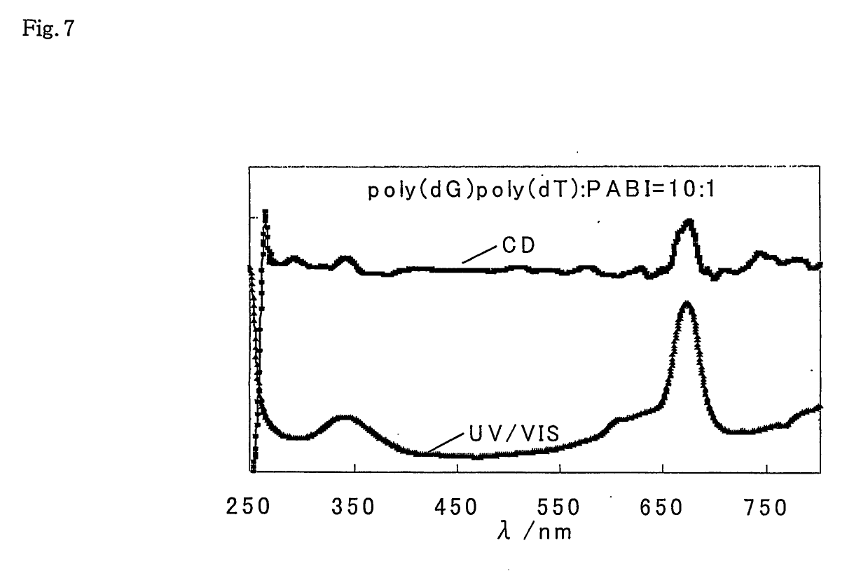

Synthetic DNA (Poly(dG) /Poly(dC), 50 mers.) used was that of Amersham Pharmacia Biotech with a concentration of 25 A₂₆₀ units and counter ions being those of a sodium salt. This DNA was adjusted to 0.25 unit with Milli-Q (registered trademark) water sterilized for 20 minutes at 120° C., and was allowed to stand for 1 day. Then, this DNA and a DMSO solution of 4×10⁻⁶ mol/L of PABI were mixed in equal amounts, allowed to stand at room temperature, and then measured for CD in the range of 250to 800 nm (see FIG. 7). In FIG. 7, a CD peak, which was not observed with PABI alone, was confirmed at a wavelength in the vicinity of 600 to 700 nm in the Q-band for PABI. Thus, it was confirmed that chirality was exhibited upon mixing of DNA and phthalocyanine.

### Effects of the Invention

According to the present invention, the inventive cationic dye compound has chirality, which is exhibited only when bound to the double strand of a nucleic acid. This dye compound can be specifically detected without being separated from a single-stranded nucleic acid or the unreacted dye compound. The cationic dye compound of the present invention has an advantage, such as that of obviating the need for a cleaning step for washing off the unreacted compound or dye compound. Based on these facts, the cationic dye compound of the present invention is useful for detecting only a double-stranded nucleic acid structure conveniently and with high sensitivity.

1. (canceled)

2. (canceled)

3. (canceled)

4. A method for detecting a hybrid nucleic acid by use of a cationic dye compound, comprising:
providing a cationic dye compound comprising a cation group and a chromophore coupled to said cation group, said chromophore having a heteropolycyclic structure containing a nitrogen atom;
bringing a nucleic acid probe and a sample containing a target nucleic acid into contact with each other under hybridization conditions to form a hybrid nucleic acid composed of said nucleic acid probe and said target nucleic acid;
binding said cationic dye compound onto said hybrid nucleic acid by adding the cationic dye compound before, during or after said hybridization; and
measuring circular dichroism of said cationic dye compound bound onto said hybrid nucleic acid.

5. The method according to claim 4, wherein said cationic dye compound is represented by the following general formula (I):
X—(Y-Z)n (I)
where n denotes 1 to 12, X represents a choromophore having at least four pyrrole rings, Y represents a connecting group or a direct bond between X and Z, and Z represents a cationic functional group, or a functional group whose property is convertible to a cationic property.

6. The method according to claim 4, wherein said choromophore is selected from the group consisting of porphyrin, porphyrin derivatives, phthalocyanine, and phthalocyanine derivatives.

7. The method according to claim 4, further comprising: immobilizing an analyte having said target nucleic acid or said nucleic acid probe onto a solid phase carrier to bring said analyte and said nucleic acid probe into contact with each other under hybridization conditions.

8. An apparatus for detecting a hybrid nucleic acid by use of a cationic dye compound, comprising:
means for bringing a nucleic acid probe and a sample containing a target nucleic acid into contact with each other under hybridization conditions to form a hybrid nucleic acid composed of said nucleic acid probe and said target nucleic acid;
means for binding said cationic dye compound onto said hybrid nucleic acid by adding the cationic dye compound before, during or after said hybridization, wherein said cationic dye compound comprises a cation group and a chromophore coupled to said cation group, said chromophore having a heteropolycyclic structure containing a nitrogen atom; and
means for measuring circular dichroism of said cationic dye compound bound onto said hybrid nucleic acid.
